(19) United States
(12) Patent Application Publication
(10) Pub. No.: US 2005/0054555 A1
(43) Pub. Date: Mar. 10, 2005

(54) Kahalalide compounds for use in cancer therapy

(76) Inventors: Jose Jimeno, Madrid (ES); Luis Lopez Lazaro, Madrid (ES); Ana Ruiz Casado, Madrid (ES); Miguel Angel Izquierdo, Madrid (ES); Jose Manuel Trigo, Malaga (ES); Jan Schellens, Kockengen (NL); Luis Paz-Ares, Madrid (ES)

Correspondence Address: FISH & RICHARDSON PC, 225 FRANKLIN ST, BOSTON, MA, 02110, US

(21) Appl. No.: 10/492,670

(22) PCT Filed: Oct. 18, 2002
(86) PCT No.: PCT/GB02/04735

Related U.S. Application Data
(60) Provisional application No. 60/348,449, filed on Oct. 19, 2001.

(51) Int. Cl. A61K 38/16

(52) U.S. Cl. 514/2

(57) ABSTRACT

Procedures for clinical trials of kahalalide compounds are provided, leading to new formulations of kahalalide compounds.

### FIELD OF THE INVENTION

The present invention relates to the use of kahalalide compounds in the treatment of cancer.

### BACKGROUND OF THE INVENTION

Nature is the origin of many effective medicines in oncology, like paclitaxel, adriamycin, etoposide, bleomycin, etc. In recent years the sea has proven to be an invaluable source for compounds displaying original chemical structures and interesting biological activity. Among the cytotoxic compounds from marine origin we can mention the ecteinascidins, didemnins, dolastatins, spisulosines, lamellarins, some of them being developed as antitumoural agents in clinical trials.

The kahalalide compounds are peptides isolated from a Hawaiian herbivorous marine species of mollusc, Elysia rufescens. Kahalalides A-F are described in EP 610 078 and Hamman et al., J. Am. Chem. Soc., 1993, 115, 5825-5826.

Kahalalide A-G are described in Hamann, M. et al., J. Org. Chem, 1996, 61, 6594-6600: “Kahalalides: bioactive peptides from a marine mollusk Elysia rufescens and its algal diet Bryopsis sp.”.

Kahalalide H and J are described in Scheuer P. J. et al., J. Nat. Prod. 1997, 60, 562-567: “Two acyclic kahalalides from the sacoglossan mollusk Elysia rufescens”.

Kahalalide O is described in Scheuer P. J. et al., J. Nat. Prod. 2000, 63(1) 152-4: A new depsipeptide from the sacoglossan mollusk Elysia ornata and the green alga Bryopsis species”.

For kahalalide K, see Kan, Y. et al., J. Nat. Prod. 1999 62(8) 1169-72 “Kahalalide K: A new cyclic depsipeptide from the hawaiian green alga bryopsis species”.

For related reports, see also Goetz et al., Tetrahedron, 1999, 55; 7739-7746: “The absolute stereochemistry of Kahalalide F”; Albericio, F. et al. Tetrahedron Letters, 2000, 41, 9765-9769: “Kahalalide B. Synthesis of a natural cyclodepsipeptide”; Becerro et al. J. Chem. Ecol. 2001, 27(11), 2287-99: “Chemical defenses of the sarcoglossan mollusk Elysia rufescens and its host Alga bryopsis sp.”.

The synthesis and cytotoxic activities of natural and synthetic kahalalide compounds is described in WO 01 58934.

Of the kahalalide compounds, kahalalide F is the most promising because of its antitumoural activity. Kahalalide F now known to have the structure:

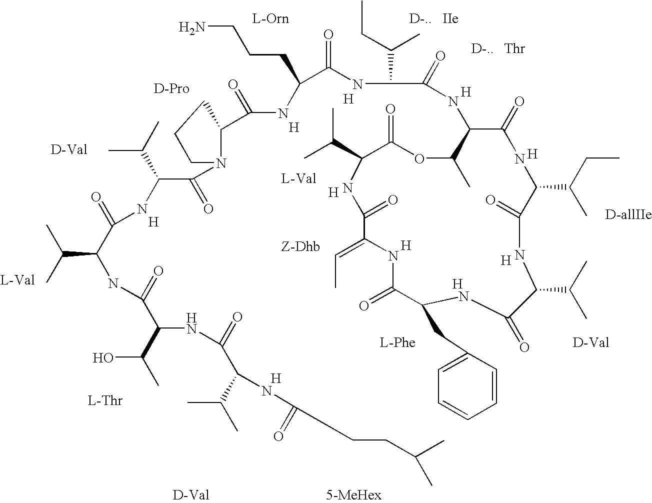

Kahalalide F is a tridecapeptide with a ring shape side and a lateral side, containing a fatty acid group connected to the latter. Its activity against in vitro cell cultures of human lung carcinoma A-549 and human colon carcinoma HT-29 were reported in EP 610 078.

WO 02 36145 describes pharmaceutical compositions containing kahalalide F and new uses of this compound in cancer therapy.

See also Beijnen, J. H. et al., Drug Dev. Ind. Pharm. 2001, 27(8) 767-80: “Development of a lyophilized parenteral pharmaceutical formulation of the investigational polypeptide marine anticancer agent kahalalide F”; Beijnen, J. H. et al., Br. J. Clin. Pharmacol. 2002, 53(5), 543: “Bioanalysis of the novel peptide anticancer drug kahalalide F in human plasma by h.p.l.c. under basic conditions coupled with positive turbo-ionspray tandem mass spectrometry”; Beijnen, J. H. et al., PDA J. Pharm. Sci. Technol. 2001, 55(4) 223-9: “In vitro hemolysis and buffer capacity studies with the novel marine anticancer agent Kahalalide F and its reconstitution vehicle cremophor EL/ethanol”; Sparidans R. W. et al., Anticancer Drugs 2001, 12(7) 575-82: “Chemical and enzymatic stability of a cyclic depsipeptide, the novel, marine-derived, anti-cancer agent kahalalide F”.

In preclinical studies, kahalalide F has shown significant activity against solid tumour cell lines, and selectivity for, but not restricted to, prostate tumour cells, neuroblastomas, some primary sarcoma lines and tumour cells that overexpress the Her2/neu oncogene. In vitro exposure studies demonstrated that kahalalide F is not schedule dependent. Its mechanism of action is not yet elucidated, in vitro studies have shown activity of kahalalide F to cause cell swelling and ultimately death, see for example Garcia-Rocha M, Bonay P, Avila J., Cancer Lett. 1996 99(1) 43-50: “The antitumoural compound Kahalalide F acts on cell lysosomes”.

Preclinical in vivo studies determined that the maximum tolerated dose (MTD) of KF in female mice following a single bolus iv injection was to be 280 μg/kg. Whereas single doses just above the MTDiv were extremely toxic, with animals exhibiting signs of neurotoxicity followed by death, 280 μg/kg KF could be administered repeatedly, according to a once daily times five schedule, without any apparent evidence of acute toxicity. See Supko, F. et al., Proceedings of the 1999 AACR NCI EORTC International Conference, abstract 315: “Preclinical pharmacology studies with the marine natural product Kahalalide F”.

During preclinical studies kahalalide F exhibited low cardiac and skeletal muscle toxicities and also low myeolotoxicity. In mice, the main toxicities found were tubular nephrotoxicity and CNS (central nervous system) neurotoxicity, and hints of hepatotoxicity. Therefore the expected toxicities were renal and neurologic with a marked threshold. As mentioned before, whereas MTD had no lethality, doses slightly over MTD showed high lethality.

It is an object of the present invention to provide new, improved forms of treatment using kahalalide compounds showing clinical benefit.

In particular, it is an object of the invention to provide dosages and schedules of kahalalide compounds that can be used for cancer therapy in humans, avoiding toxicities while maintaining the desired antineoplastic effects.

It is another object of the present invention to provide new uses in cancer therapy for the kahalalide compounds, in particular for kahalalide F.

It is yet another object of the invention to provide new products containing kahalalide compounds, in particular kahalalide F, for administration in the treatment of cancer.

### SUMMARY OF THE INVENTION

We have developed a method to treat human patients with kahalalide compounds, in particular kahalalide F, avoiding toxicity and leading to clinical improvement.

The present invention provides a method for treating a human patient afflicted with cancer, comprising administering to said patient a therapeutically effective amount of kahalalide compound, or a pharmaceutical composition thereof. More preferably the kahalalide compound is kahalalide F.

We have found that contrary to what was expected, the dose limiting toxicity in the treatment with a kahalalide compound is liver toxicity with grade 4 transaminase elevation. This toxicity is asymptomatic, manageable and reversible if the proper dosages and schedules are selected. Transaminases peak 4-5 hours after kahalalide infusion.

The present invention provides a pharmaceutical composition containing a recommended dose of a kahalalide compound and a pharmaceutically acceptable carrier.

In particular, the present invention provides a procedure for establishing the amount of a kahalalide compound to be recommended for dosing to patients, which procedure comprises administering the compound in a series of escalating doses to a cohort of humans, monitoring for transaminase elevation as the dose-limiting toxicity, determining a maximum tolerated dose, and establishing a recommended dose.

Such a procedure can be used in clinical trials. The maximum tolerated dose is suitably set as one in which a proportion of the cohort encounter dose-limiting toxicity. For example, the proportion is typically 2 out of 6. The recommended dose can then be established in accordance with familiar principles. Usually the recommended dose is the dose below the maximum tolerated dose, but sometimes rules are applied regarding the proportion of the cohort which encounter dose-limiting toxicity at the proposed recommended dose.

The procedure for clinical trials enables a method of preparing a pharmaceutical composition containing a kahalalide compound, the method comprising carrying out the procedure to establish the recommended dose, and formulating the kahalalide compound with a pharmaceutically acceptable carrier to give a dosage form which contains the recommended dose of the kahalalide compound.

The invention further provides for the use of kahalalide compounds in the preparation of a composition for the procedures and methods of this invention.

In one aspect, the present invention provides a method for treating a human patient afflicted with cancer, comprising administering to said patient a kahalalide compound at a dose below 1200 mcg/m2/day, preferably below 930 mcg/m2/day and more preferably below 800 mcg/m2/day. Suitably the dose is at least 320 mcg/m2/day. Preferably the dose is in the range of 400-900 mcg/m2/day, preferably 500-800 mcg/m2/day, more preferably 600-750 mcg/m2/day. Especially preferred are doses of about 650-700 mcg/m2/day.

We have found that the selected schedule is important to allow for a reversion of the liver toxicity. If a daily 1 hour intravenous infusion is used during 5 days, the dose limiting toxicity is reached at 930 mcg/m2/day. Thereafter a rest period of 2 weeks is needed to reverse the toxicity effects.

Therefore in one aspect the invention provides a method for treating a human patient afflicted with cancer, comprising administering to said patient a kahalalide compound daily during 5 days at a dose below 930 mcg/m2/day, followed by a resting period of from 1 to 4 weeks in which the kahalalide compound is not administered. The dose is preferably 650-750 mcg/m2/day, more preferably about 700 mcg/m2/day. The infusion time is preferably between 1 and 24 hours, more preferably between 1 and 3 hours. Especially preferred is an infusion time of about 1 hour. The resting period is preferably 2-3 weeks, more preferably about 2 weeks.

Surprisingly for this kind of cancer therapy, we also found that a weekly schedule is possible without resting period if the dosage is about 650 mcg/m2/day. Transaminases elevation is reversible by day 8th at about 650 but not at >800. In this case the liver toxicity was found to be reversible within one week and no resting period is needed, with the obvious advantages this supposes.

Therefore, in another aspect the present invention provides a method for treating a human patient afflicted with cancer, comprising administering to said patient a kahalalide compound once weekly at a dose below 800 mcg/m2/day. The dose is preferably 600-700 mcg/m2/day, more preferable 650 mcg/m2/day. The infusion time is preferably between 1 and 24 hours, more preferably between 1 and 3 hours. Especially preferred is an infusion time of about 1 hour.

The above schedules and dosages allow for an effective cancer therapy in humans. We have found that kahalalide compounds, and in particular kahalalide F is effective in the treatment of advanced solid tumours (AST), including metastatic tumours. Tumours that are preferably treated are hormone independent prostate cancer, hepatocarcinoma, epithelial carcinomas, non small cell lung cancer and mesothelioma.

In a further aspect of the present invention, a medical kit for administering a kahalalide compound is provided, comprising printed instructions for administering the kahalalide compound according to the dosing schedules set forth above, and a supply of kahalalide compound in dosage units for at least one cycle, wherein each dosage unit contains the appropriate amount of kahalalide compound for the treatments as defined above and a pharmaceutically acceptable carrier.

### DETAILED DESCRIPTION

The term “kahalalide compound” includes natural compounds, their mixtures and new compounds as defined in WO 01 58934 which is incorporated herein in its entirety by reference. Especially preferred is the compound kahalalide F.

Thus, the present invention employs a natural kahalalide such as kahalalide F or a mimic of a natural kahalalide. The mimic compounds may differ in one or more amino acids, and one or more components of the acyl side chain. Preferably they differ in one or more components of the acyl side chain. Examples of the kahalalide compound for use in this invention particularly include the compound identified as kahalalide F with a 5-methylhexyl sidechain, compounds differing only in the sidechain such as the 4-methylhexyl analogue, and mixtures thereof.

Suitably the mimics have at least one of the following features to differentiate from a parent naturally occurring kahalalide:
1 to 7, especially 1 to 3, more especially 1 or 2, most especially 1, amino acid which is not the same as an amino acid the parent compound;
1 to 10, especially 1 to 6, more especially 1 to 3, most especially 1 or 2, additional methylene groups in the side chain acyl group of the parent compound;
1 to 10, especially 1 to 6, more especially 1 to 3, most especially 1 or 2, methylene groups omitted from the side chain acyl group of the parent compound;
1 to 6, especially 1 to 3, more especially 1 or 3, substituents added to or omitted from the side chain acyl group of the parent compound.

For cyclic kahalalides, the amino acid addition or omission can be in the cyclic ring or in the side chain.

Examples of mimic comopunds are compounds related to kahalalide F, and having the formula: 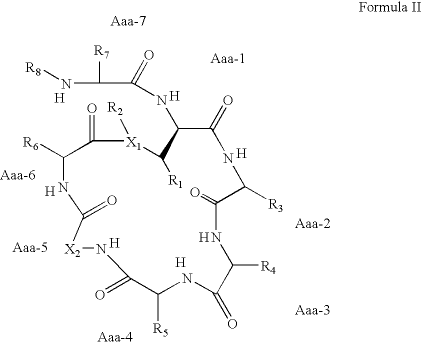
wherein Aaa₁, Aaa₂, Aaa₃, Aaa₄, Aaa₆, and Aaa₇ are independently α-amino acids of L or D configuration, if applies; wherein R₁, R₂, R₃, R₄, R₅, R₆, R₇ are each independently H or an organic group selected from the group consisting of an alkyl group, an aryl group, an aralkyl group, and their substituted derivatives with an hydroxy group, a mercapto group, an amino group, a guanidino group, a halogen group; wherein X₁ is independently O, S, or N; wherein R₂ is, if applies, independently H or an organic group selected from the group consisting of an alkyl group and an aralkyl group; wherein Aaa₅ is independently an amino acid of L or D configuration, if applies; wherein X₂ is independently an organic group selected from the group consisting of an alkenyl, an alkyl group, an aryl group, an aralkyl group, and their substituted derivatives with an hydroxy group, a mercapto independently H or an organic group selected from the group consisting of an alkyl group, an aryl group, an aralkyl group, and their substituted derivatives with an hydroxy group, a mercapto group, an amino group, a guanidino group, a halogen group; wherein R₈ is independently of the following formulae III, IV, or V: 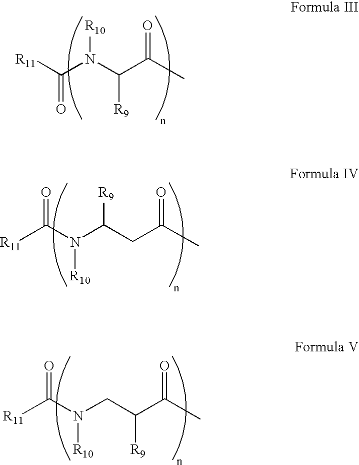
wherein R₉, R₁₀, and R₁₁ are each independently H or an organic group selected from the group consisting of an alkyl group, an aryl group, an aralkyl group, and their substituted derivatives with an hydroxy group, a mercapto group, an amino group, a guanidino group, a carboxyl group, a carboxamido group, a halogen group; R₉ and R₁₀ can form part of the same cycle; R₉ can confer S or R configuration, if applies, to the carbon attached to; and n is 0 to 6. The definitions of the amino acids can also be varied to allow for proline and analogous amino acids including hydroxyproline. The formulae (III), (IV) and (V) can be intermixed to give a side chain made up of repeat units in more than one of these formulae.

In a modification, one or more of the ring amino acids Aaa-6 and Aaa-5 of the hexaamino acid cycle is omitted or an amino acid Aaa-7 is added between Aaa-6 and Aaa-1, in order to arrive at rings having four, five or seven ring amino acids. Six ring amino acids is preferred.

Depending on the type of tumour and the developmental stage of the disease, the treaments of the invention are useful in preventing the risk of developing tumours, in promoting tumour regression, in stopping tumour growth and/or in preventing metastasis.

Administration of the compounds or compositions of the present invention is by intravenous infusion. Infusion times of up to 72 hours can be used, more preferably 1 to 24 hours, with either about 1 or about 3 hours most preferred. Short infusion times which allow treatment to be carried out without an overnight stay in hospital are especially desirable. However, infusion may be around 24 hours or even longer if required.

Although guidance for the dosage is given above, the correct dosage of the compound will vary according to the particular formulation, the mode of application, and the particular situs, host and tumour being treated. Other factors like age, body weight, sex, diet, time of administration, rate of excretion, condition of the host, drug combinations, reaction sensitivities and severity of the disease shall be taken into account. Administration can be carried out continuously or periodically within the maximum tolerated dose.

The Recommended Dose (RD) is the highest dose which can be safely administered to a patient producing tolerable, manageable and reversible toxicity according to the Common Toxicity Criteria (CTC) established for example by the National Cancer Institute, (USA) typically with no more than 2 out of 6 patients presenting any dose limiting toxicities (DLT). Guidelines for cancer therapy frequently call for administration of chemotherapeutic agents at the highest safe dose at which toxicity is manageable in order to achieve maximum efficacy (DeVita, V. T. Jr., Hellman, S. and Rosenberg, S. A., Cancer: Principles and Practice of Oncology, 3rd ed., 1989, Lipincott, Philadelphia). For the kahalalide compounds, in particular kahalalide F, the recommended doses are as defined above and set forth in the examples.

The administration is performed in cycles, in the preferred application method, an intravenous infusion of kahalalide compounds given to the patients the first week of each cycle, the patients are allowed to recover for the remainder of the cycle. The preferred duration of each cycle is of either 1, 3 or 4 weeks; multiple cycles can be given as needed. Dose delays and/or dose reductions and schedule adjustments are performed as needed depending on individual patient tolerance of treatments, in particular dose reductions are recommended for patients with higher than normal serum levels of liver transaminases or alkaline phosphatase.

Pharmaceutical compositions of kahalalide compound that can be used include liquid (solutions, suspensions or emulsions) with suitable composition for intravenous administration, and they may contain the pure compound or in combination with any carrier or other pharmacologically active compounds. Further guidance concerning the pharmaceutical compositions can be found in WO 02 36145 which is incorporated herein by reference in its entirety.

Thus, a combination of a non-ionic surfactant and an organic acid is suited for use with a bulking agent to give a lyophilised form of a kahalalide compound suited for reconstitution. Reconstitution is preferably effected with a mix of emulsifying solubiliser, alkanol and water.

The lyophilised composition preferably comprises mainly the bulking agent, such as at least 90% or at least 95% bulking agent. Examples of bulking agents are well known and include sucrose and mannitol. Other bulking agents can be employed.

The non-ionic surfactant in the lyophilised composition is preferably a sorbitan ester, more preferably a polyethylene sorbitan ester, such as a polyoxyethylene sorbitan alkanoate, especially a polyoxyethylene sorbitan mono-oleate, for example polysorbate 80. The non-ionic surfactant typically comprises a few % of the composition, such as 0 to 5% of the composition, for instance 2 to 3 or 4% of the composition.

The organic acid in the lyophilised composition is typically an aliphatic acid, preferably a hydroxycarboxylic acid and more preferably a hydroxypolycarboxylic acid, notably citric acid. The organic acid typically comprises a few % of the composition, such as 0 to 5% of the composition, for instance 2 to 3 or 4% of the composition.

The amount of kahalalide compound in the lyophilised composition is typically less than 1%, or often less than 0.1%, of the mix. A suitable amount is in the range 50 to 200 μg, say about 100 μg, per 100 mg of composition.

The emulsifying solubiliser for the reconstituting agent suitably comprises an polyethylene glycol ester, notably an ester of a fatty acid, more preferably a PEG oleate such as PEG-35 oleate. The emulsifying solubiliser is suitably 0 to 10% of the reconstituting agent, typically about 3 to 7%, say about 5%. The alkanol is usually ethanol, and is suitably 0 to 10% of the reconstituting agent, typically about 3 to 7%, say about 5%. The remainder of the reconstituting agent is water, and gives a reconstituted solution suited for intravenous injection.

Further dilution of the reconstituted solution with 0.9% saline may be appropriate for infusion of the kahalalide compound.

In a particularly preferred embodiment, the lyophilised composition comprises 150 μg kahalalide F; 150 mg sucrose; 3 mg anhydrous citiric acid; and 3 mg of polysorbate 80.

The preferred reconstituting agent then comprises 2 to 7%, say about 5%, emulsifying solubiliser; 2 to 7%, say about 5%, alcohol; and remainder water.

The invention additionally provides kits comprising separate containers containing the lyophilised composition and the reconstituting agent. Methods of reconstitution are also provided.

The present invention further provides a method of treating any mammal, notably a human, affected by cancer which comprises administering to the affected individual a therapeutically effective amount of a pharmaceutical composition thereof prepared by reconstitution of a lyophilised composition of this invention. The present invention can be employed particularly for treatment of patients with refractory cancers that do not respond favourably to other treatments. In particular, the compositions of this invention can be employed after other chemotherapy has been tried and not worked.

In one embodiment, the reconstituted solution is prepared for infusion and is administered in a 3-hour infusion on concentrations of up to around 20 or 25 μg/ml, typically up to 15 μg/ml. Suitable infusion equipment preferably includes a glass container, rather than one of polyethylene. Tubing is preferably of silicone.

We prefer that infusion times of up to 24 hours are used, and as explained we prefer an infusion time of about 1 hour. In a variation, the infusion time is 2-12 hours, such as 2-6 hours. Short infusion times which allow treatment to be carried out without an overnight stay in hospital are especially desirable. However, infusion may be 12 to 24 hours or even longer if required. Infusion may be carried out at suitable intervals of say 2 to 4 weeks. In an alternative dosing protocol, the kahalalide compound such as kahalalide F is administered for say about 1 hour for 5 consecuvitve days every 3 weeks. Other protocols can be devised as variations.

The compounds and compositions of this invention may be used with other drugs to provide a combination therapy. The other drugs may form part of the same composition, or be provided as a separate composition for administration at the same time or a different time. The identity of the other drug is not particularly limited.

### EXAMPLES OF THE INVENTION

Clinical trials were carried out based on the following protocols:

Number of Patients Per Dose Level and Dose Escalation

The rate of subject entry and escalation to the next dose level will depend upon assessment of the safety profile of patients entered at each dose level. Toxicity will be evaluated and graded according to the Common Toxicity Criteria, version 2.0.

In order to minimize the number of patients treated at the subtoxic dose levels, a single patient will be treated per dose level with 100% dose escalation in the absence of any toxicity G2 (excluding alopecia) during their first course of treatment. After the occurrence of grade 2 toxicity (excluding asthenia, alopecia, nausea and vomiting or anemia), the dose level is expanded to three patients with only 50% incremental increase in dosage in advancing to the next dose level. For the occurrence of grade 3 non-hematological toxic effects the dose level should be expanded up to six patients. This second cohort of 3 patients will be included consecutively. If the first or second patient develops dose limiting toxicities no further patients will be treated at this dose level, it will be defined as MTD and the accrual will begin at a lower dose level. If only one DLT occurs at this dose level another 3 patients will be included at the next dose level with an increase of 25% only.

| Observed toxicities | Action |
|---|---|
| <G1 toxicities | Treat 1 patient per dose level with |
| | 100% dose escalation |
| G2 toxicities | Treat 3 pats at that dose level with |
| | 50% increase in Dose escalation |
| G3 toxicities non- | Expand current dose level up to 6 |
| hematological | pats and 25% increase in dose |
| | escalation |
| ≧⅓ or ≧2/6 DLTs | MTD |

Clinical pharmacokinetic data on Kahalalide F will become available during the course of each dose level and will be considered when making a final judgement.

There is no intrapatient dose escalation, only de-escalation. If the toxicity is grade 3 or worse in a course of chemotherapy, then the dose is reduced one level for the next course.

Maximum Tolerated Dose (MTD) and Recommended Dose (RD) for Phase II Studies

The maximum tolerated dose (MTD) is defined as the dose at which at least 2 out of 3 or more than 3 patients experience DLT. However, it is possible that additional patients may experience DLT due to the timing of patient enrollment into that dose level.

Once an MTD level is established, subsequent patients should be treated at the next lower dose level. Intermediate doses may be used in some instances and flexibility is an integral part of the protocol.

At the RD, sufficient number of patients will be accrued to further define the toxicity profile at this dose level.

KHF A 001-00

Definition of Dose Limiting Toxicities

Dose limiting toxicities (DLTs) are defined as follows:

Grade IV Neutropenia lasting for >5 days or with a temperature >38.5.

Grade IV thrombocytopenia.

Any other grade III or IV non hematological toxicity, excluding nausea, emesis and hypersensitivity reactions.

Grade III or IV liver toxicity if not reversible by day 21.

These toxicities are only considered DLTs if they happen during cycle 1.

KHF 002-01

Definition of Dose Limiting Toxicities

The toxicity will be evaluated and clasified in grades according to the Common Toxicity Criteria (CTC) version NCI 2.0, March 1998.

Dose Limiting Toxicities (DLTs) are Defined as Follows:

ANC <500/mm3 for more than 5 days.

ANC <500/mm3 with a temperature of 38'5 or more.

Plaquetas <25000/mm3

Any other non-hematological grade 3-4 toxicity excluding: nausea, emesis without prophilaxis

G3 transaminases increase >14 days

Hypersensibility reactions

### Example 1

Preliminary Report

Phase I and Pharmacokinetic Study of Kahalalide F in Patients With Advanced Androgen Refractory Prostate Cancer

INTRODUCTION: Kahalalide F (KF) is one of a family of novel dehydroaminobutyric acid-containing peptides isolated from the Hawaiian herbivorous marine species of mollusk, Elysia rufescens. KF displays both in vitro and in vivo anti-tumour activity in various solid tumour models including breast, colon, non-small cell lung, and in particular prostate cancer. On the basis of its selectivity, KF is now further developed as a potential anticancer agent against androgen independent prostate tumours. OBJECTIVE: In the present phase I clinical and pharmacokinetic (PK) study the toxicity profiles PK and anti-tumour activity of KF are investigated. METHODS: KF is administered as an intravenous infusion over one hour, during five consecutive days every three weeks in patients with advanced or metastatic androgen refractory prostate cancer. On the basis of the MTD values defined in mice, a starting dose of 20 μg/m2/day was selected, which is equivalent to a total dose of 100 μg/m2. PK of KF were determined in plasma during the first course. Bioanalysis of KF was performed by LC-MS/MS. LDH, AF and especially PSA levels of each patient were also evaluated during the study to determine the activity of KF. RESULTS AND DISCUSSION: At present 7 patients have been registered. Patients had a median age of 66 years (range 54-75). One patient per level was entered at 20, 40, 80 and 160 μg/m2/day. Due to transaminase elevation a number of 4 patients were entered at the current dose level, 320 μg/m2/day. The first patient of this study was re-entered at this dose level. Observed adverse events were rapidly reversible mild headache, fatigue, pain and local edema. The only drug related toxicity to date was a rapidly reversible CTC grade 3 ASAT that occurred at 320 μg/m2/day. PK revealed a linear relationship between dose and AUC over the whole dose range. Total plasma clearance was 267 mL/min (±115) and the terminal half-life of intravenous KF in these patients was 0.46 h (±0.13). Maximum plasma concentrations reached at the current dose level (35-50 ng/mL) are potentially active for prostate cancer in the clonogenic tumour assay (activity from 15 ng/mL). Thus far, the schedule is well tolerated. One patient sowed a significant decrease in PSA level (>50%) associated to clinical improvement (pain relief). Two additional patients experienced minor PSA reductions, one still ongoing after two cycles. The maximum tolerated dose has not been reached yet and the study is ongoing.

### Example 2

KHF-A-001-00. Phase I Clinical and Pharmacokinetic study to determine the safety of kahalalide F administered as a daily x 5 over 1 hour Infusion every 21 days in patients with advanced or metastatic prostate cancer.

The first clinical trial with KHF is being run in The Netherlands. It is addressed to prostate cancer patients according to the high selectivity exhibited in the preclinical programme. The toxicity data in rodents and dogs shows the feasibility to give daily doses equivalent to the single dose MTD in a repeated fashion (daily times five): such schedule may significantly enhance the therapeutic profile of KF in patients bearing hormone resistant prostate cancer.

A dose of 20 mcg/m2 was considered a safe starting dose on the basis of acute toxicity studies in animals. The trial was designed as an accelerated dose escalation (pharmacokinetically guided).

One patient (the first one treated in this trial) received 4 cycles at the first dose level. 2 months after stopping the treatment, was included at the current dose level (V) and received another 4 cycles. Grade 3 hypertransaminasemia was the reason to expand the cohort in the 5th level.

| | Level | | | | | | | | | |
|---|---|---|---|---|---|---|---|---|---|---|
| | I | II | III | IV | V | VI | VII | VIII | IX | X |
| Dose | 20 | 40 | 80 | 160 | 320 | 425 | 560 | 700 | 930 | 700 |
| Increase % | | 100 | 100 | 100 | 100 | 33 | 33 | 25 | 33 | |
| Patient | 1 | 1 | 1 | 1 | 5 | 3 | 3 | 3 | 3 | 2 |
| Cycles | 4 | 1 | 8 | 4 | 2, 4, 8, 2, 2 | 3, 2, 2 | 4, 2, 2 | 2, 2, 2 | 5, 3, 2 | |
| Worst | none | none | none | none | G3 | none | G3 | none | G4 | |
| toxicity | | | | | Hypertrans | | Hypertrans | | Hypertrans | |
| | | | | | | | | | Hypersensitivity | |
| Efficacy | | | PR | SD | | | SD | | | |

20 patients and 66 cycles could be evaluated by August, 2002.

HEMATOLOGIC TOXICITY % (worst per patient)

| 19 patients | Grade 1 | Grade 2 | Grade 3 | Grade 4 |
|---|---|---|---|---|
| Neutrophils | 5 | 5 | 0 | 0 |
| Leukocytes | 10.5 | 0 | 0 | 0 |
| Platelets | 21.1 | 0 | 0 | 0 |
| Hemoglobin | 63.2 | 26.3 | 0 | 0 |

NON-HEMATOLOGIC TOXICITY % LAB
ABNORMALITIES per patient

| 20 patients | Grade 1 | Grade 2 | Grade 3 | Grade 4 |
|---|---|---|---|---|
| Creatinine | 35 | 0 | 0 | 0 |
| Bilirubin | 10 | 0 | 0 | 0 |
| Alk. phosphatase | 35 | 20 | 25 | 5 |
| GOT | 40 | 15 | 15 | 10 |
| GPT | 15 | 25 | 20 | 10 |
| GGT | 35 | 15 | 25 | 5 |
| HypoNa+ | 10 | 0 | 5 | 5 |
| HypoK+ | 20 | 0 | 0 | 0 |
| HypoCa* | 65 | 0 | 0 | 0 |
| Hypo P | 10 | 15 | 0 | 0 |
| Albumin | 30 | 10 | 5 | 0 |
| CPK | 35 | 0 | 0 | 0 |

*Calcium adjusted

Most frequently reported NON-HEMATOLOGIC TOXICITIES

| 20 patients | Grade 1 | Grade 2 | Grade 3 | Grade 4 |
|---|---|---|---|---|
| Diarrhea | 10 | 5 | 0 | 0 |
| Fatigue | 35 | 10 | 25 | 0 |
| Nausea | 15 | 10 | 0 | 0 |
| Hypersensitivity | 20 | 5 | 0 | 0 |
| Hypertension | 0 | 0 | 15 | 0 |
| Inject-site | 10 | 20 | 0 | 0 |
| reaction | | | | |
| Periph-sens | 20 | 0 | 5 | 0 |
| neuro | | | | |
| Vomiting | 15 | 10 | 0 | 0 |

From preclinical studies renal and neurologic were the most expected toxicities, but surprisingly none of them has constituted remarkable toxicity. However, liver toxicity was the most commonly seen as grade 4 transaminases elevation in the MTD (GOP, GPT levels). Patients also complain of puncture or prick on hands palms during the infusion.

Hypertension and alkaline phosphatase were not drug-related.

As it was predicted by preclinical experiments no hematological toxicity was seen. No grade 3-4 emesis was reported.

Regarding efficacy, one patient was evaluated as a partial response (evaluated by PSA). This patient received 8 cycles. Two more patients exhibited SD (they were also evaluated by PSA) as best response and received 4 cycles. All these patients concomitantly showed clinical improvement.

### Example 3

KHF-A-002-01. Phase I Clinical and Pharmacokinetic study to determine the safety of Kahalalide F administered as a weekly infusion over 1 hour in patients with solid tumours.

This trial was addressed to any solid tumours and was designed as a classical escalation. The starting dose was higher because we had some information from the first trial, and this allowed us to skip the first steps. One cycle in this trial means a week, so 4 cycles mean 1 month of treatment.

The second dose level (400) was expanded because of two unrelated adverse events: grade 3 diarrhea and death due to gastrointestinal bleeding. This was also the reason why the next escalation was only 32.5% instead of 50%. No toxicities were reported in this level and the following escalation was 50% again.

The DLT was identified at 1200 mcg/m2, and was grade 4 hypertransaminasemia non reversible by day 21. The time of onset was located at 5 hours after the infusion. This was not a scheduled determination and that was why the dose was descalated looking for previously unidentified grade 4 hypertransaminasemia. The 4th level was reexplored and this second time, grade 4 hypertransaminasemia was identified as DLT again at this dose level.

19 patients and 145 cycles were evaluated.

| | Level | | | | | | | |
|---|---|---|---|---|---|---|---|---|
| | I | II | III | IV | V | VI | VII | VIII |
| Dose | 266 | 400 | 530 | 800 | 1200 | 1000 | 800 | 650 |
| Increase % | | | | | | | | |
| Patient | 3 | 6 | 3 | 3 | 5 | 3 | 3 | 10 |
| Cycles | 5, 8, 8 | 2, 6, 7, | 7, 8, 16 | 10, 11, 8 | 8, 8, 3, 8 | 3, 1, 3 | 2, 1, 1 | |
| | | 9, 21, 4 | | | | | | |
| Worst | — | | | | Hypersensitivity | Hypersensitivity | G4 | |
| toxicity | | | | | hypertrans | hypertrans | Hypertrans | |
| Efficacy | — | Cavum | NSCLC | | | | | |
| | | Hepatocell | | | | | | |

Patients have complained of puncture or prick on hands palms in all the dose levels.

One SAE with hypersensitivity reactions was reported. The patient had both pruritus and bronchoconstriction after the first cycle at 1200 mcg/sm. He could not be retreated in spite of administering standard premedication.

Other Toxicities

HEMATOLOGIC TOXICITY (worst per patient)

| | Grade 1 | Grade 2 | Grade 3 | Grade 4 |
|---|---|---|---|---|
| Neutrophils | 5.3 | 5.3 | 0 | 0 |
| Leukocytes | 15.8 | 5.3 | 0 | 0 |
| Platelets | 10.5 | 0 | 0 | 0 |
| Hemoglobin | 57.9 | 21.1 | 0 | 0 |

NONHEMATOLOGIC TOXICITY.
LABORATORY ABNORMALITIES

| | Grade 1 | Grade 2 | Grade 3 | Grade 4 |
|---|---|---|---|---|
| Creatinine | 31.6 | 5.3 | 0 | 0 |
| Bilirubin | 5.6 | 0 | 0 | 0 |
| Alk. phosphatase | 27.8 | 11.1 | 0 | 0 |
| GOT | 38.9 | 0 | 0 | 16.7 |
| GPT | 22.2 | 5.6 | 0 | 16.7 |
| GGT | 16.7 | 11.1 | 5.6 | 11.1 |
| Na+ hypo | 21.1 | 0 | 0 | 5.3 |
| K+ hypo | 5.3 | 0 | 0 | 0 |
| Ca++ hypo | 21.1 | 0 | 0 | 0 |
| Ca++ hyper | 84.2 | 0 | 0 | 0 |
| CPK | 36.8 | 0 | 0 | 0 |

Most frequently reported NON-HEMATOLOGIC TOXICITIES

| 20 patients | Grade 1 | Grade 2 | Grade 3 | Grade 4 |
|---|---|---|---|---|
| Diarrhea | 0 | 5.3 | 5.3 | 0 |
| Fatigue | 21.1 | 21.1 | 0 | 0 |
| Nausea | 31.6 | 10.5 | 0 | 0 |
| Hypersensitivity | 15.8 | 5.3 | 5.3 | 0 |
| Hypertension | 0 | 0 | 0 | 0 |
| Inject-site | 0 | 5.3 | 0 | 0 |
| reaction | | | | |
| Periph-sens | 36.8 | 0 | 5.3 | 0 |
| neuro | | | | |
| Vomiting | 10.5 | 5.3 | 0 | 0 |

Efficacy

Some hints of efficacy were seen in this trial, in a patient with a epidermoid carcinoma of the cavum, treated at 400 mcg/m2. Subjective improvement and non evaluable response (ORL exploration). Another patient with hepatocellular carcinoma, treated at 400 mcg/m2. He received the treatment during 6 months (24 cycles). Partial response in one target lesion (overall response: stable disease). Also in a patient with non small cell lung cancer, treated at 530 mcg/m2. He received the treatment during 4 months (16 infusions) without evidence of progression disease. One patient with peritoneal mesothelioma had clinical benefit with reduction of ascytes volume.

Hematologic Toxicity

KHF was supposed to be a non myelosuppressor agent. This prediction seems to have been confirmed in the clinical programme. Grade 1-2 neutropenia was seen in 10% of patients. No grade 3-4 toxicities were reported.

Renal Toxicity

This was an expected toxicity for KHF since it was dose limiting toxicity in the preclinical programme. However renal toxicity has not been a relevant toxicity. Only about 35% of patients exhibited grade 1 renal toxicity. Since this renal toxicity was expected to be more tubular than glomerular, we have carefully reviewed seric ions. Grade 1 hypokalyemia occurred in the 20% of patients, grade 1 and 2 hypophosphatemia in 10 and 15%. Grade 1 hypocalcemia was a frequent feature in the first trial (patients with prostate cancer) but it was not so frequent in patients treated in the second trial. Ions loss in urine should be confirmed.

Hypertransaminasemia

It has been the DLT for KHF. It is clearly dose-related. At 800-1200 mcg/m2/wk all the patients had grade 4 hypertransaminasemia. Hypertransaminasemia peaks 4-5 hours after the infusion of KHF and is not reversible by day 8 for patients treated at 1000 and 1200. One patient showed LDH elevation too. Neither bilirubin nor alkaline phosphatase elevations were reported. Patients remained asymptomatic. This toxicity was completely reversible.

Though transaminases are found in other organs, a grade 4 ALT is a marker of hepatocellular injury.

Hipersensitivity

Probably the most frequent toxicity observed with KHF is the palm hand prick referred by patients while the infusion. Sometimes this complaint was accompanied by other symptoms like erythema or pruritus in other locations.

Bronchoconstriction was observed in two patients at the highest dose level (1000 and 1200). Pruritus with redness and sometimes facial edema was seen. It could be more a toxic effect than a immune mediated (hypersensitivity reaction) sine it was more commonly reported in the highest levels.

### CONCLUSIONS

In this second example dose limiting toxicity for Kahalalide F was grade 4 hypertransaminasemia as in the previous example with a different schedule. It is an asymptomatic and reversible feature that appears 4-5 hours after the infusion of KHF. Hypersensitivity reactions with cutaneous features were commonly reported at highest levels.

Lack of hematologic toxicity was confirmed as it was predicted by preclinical studies.

1. A pharmaceutical composition containing a recommended dose of a kahalalide compound and a pharmaceutically acceptable carrier.

2. A procedure for establishing the amount of a kahalalide compound to be recommended for dosing to patients, which procedure comprises administering the compound in a series of escalating doses to humans, monitoring for transaminase elevation as the dose-limiting toxicity, determining a maximum tolerated dose, and establishing a recommended dose.

3. A method of preparing a pharmaceutical composition containing a kahalalide compound, the method comprising carrying out the procedure according to claim 2 to establish the recommended dose, and formulating the kahalalide compound with a pharmaceutically acceptable carrier to give a dosage form which contains the recommended dose of the kahalalide compound.

4. A composition of a kahalalide compound containing a recommended dose determined by a procedure according to claim 2.

5. (Canceled)

6. The method for treating cancer in a human patient comprising administering a kahalalide compound at a recommended dose to the subject.

7. A method of claim 6, wherein the recommended dose is established by a procedure administering the compound in a series of escalating doses to humans, monitoring for transaminase elevation as the dose-limiting toxicity, determining a maximum tolerated dose, and establishing a recommended dose.

8. A composition according to claim 1 wherein the recommended dose is less than the amount of the kahalalide compound which causes a grade 4 transaminase elevation.

9. A composition according to claim 1, which comprises a lyophilised mix of a kahalide compound, a non-ionic surfactant, an organic acid and a bulking agent.

10. A kit comprising a formulation of claim 9, together with instructions for dilution with a reconstitution solution of a mix of emulsifying solubiliser, alkanol and water.

11. A method of treating a human with a kahalalide compound which comprises administering at least 320 mcg/m2/day.

12. A method according to claim 11, for treating a human patient afflicted with cancer, comprising administering to said patient a kahalalide compound at a dose below 1200 mcg/m2/day.

13. A method according to claim 12, wherein the dose is below 930 mcg/m2/day.

14. A method according to claim 12, wherein the dose is below 800 mcg/m2/day.

15. A method according to claim 11, wherein the dose is in the range of 400-900 mcg/m2/day.

16. A method according to claim 15, wherein the dose is 500-800 mcg/m2/day.

17. A method according to claim 15, wherein the dose is 600-750 mcg/m2/day.

18. A method of administering a kahalalide compound to a human, wherein the compound is adminstered according to a schedule which is selected to allow for a reversion of liver toxicity.

19. A method for treating a human patient afflicted with cancer, comprising administering to said patient a kahalalide compound daily during 5 days at a dose below 930 mcg/m2/day, followed by a resting period of from 1 to 4 weeks in which the kahalalide compound is not administered.

20. A method according to claim 19, wherein the dose is 650-750 mcg/m2/day.

21. A method according to claim 19, wherein the dose is about 700 mcg/m2/day.

22. A method according to claim 19, wherein the infusion time is 1 to 24 hours.

23. A method according to claim 22, wherein the infusion time is 1 to 3 hours.

24. A method according to claim 23, wherein the infusion time is about 1 hour.

25. A method according to claim 19, wherein the resting period is 2-3 weeks.

26. A method according to claim 25, wherein the resting time is about 2 weeks.

27. A method for treating a human patient afflicted with cancer, comprising administering to said patient a kahalalide compound once weekly at a dose below 800 mcg/m2/day.

28. A method according to claim 27, wherein the dose is 600-700 mcg/m2/day.

29. A method according to claim 28, wherein the dose is 650 mcg/m2/day.

30. A method according to claim 27, wherein the infusion time is 1 to 24 hours.

31. A method according to claim 30, wherein the infusion time is 1 to 3 hours.

32. A method according to claim 31, wherein the infusion time is about 1 hour.

33. A method according to claim 11, wherein the kahalalide compound is kahalalide F.

34. A method according to claim 11, wherein the human has a metastatic or other advanced solid tumour.

35. A method according to claim 11, wherein the human has a hormone independent prostate cancer, hepatocarcinoma, epithelial carcinoma, non small cell lung cancer or mesothelioma.

36. (Canceled)

37. A procedure according to claim 2, wherein the recommended dose is less than the amount of the kahalalide compound which causes a grade 4 transaminase elevation.

38. A method according to claim 3, wherein the recommended dose is less than the amount of the kahalalide compound which causes a grade 4 transaminase elevation.

39. A method according to claim 18, wherein the kahalalide compound is kahalalide F.

40. A method according to claim 19, wherein the kahalalide compound is kahalalide F.

41. A method according to claim 27, wherein the kahalalide compound is kahalalide F.

42. A method according to claim 18, wherein the human has a metastatic or other advanced solid tumour.

43. A method according to claim 19, wherein the human has a metastatic or other advanced solid tumour.

44. A method according to claim 27, wherein the human has a metastatic or other advanced solid tumour.

45. A method according to claim 18, wherein the human has a hormone independent prostate cancer, hepatocarcinoma, epithelial carcinoma, non small cell lung cancer or mesothelioma.

46. A method according to claim 19, wherein the human has a hormone independent prostate cancer, hepatocarcinoma, epithelial carcinoma, non small cell lung cancer or mesothelioma.

47. A method according to claim 27, wherein the human has a hormone independent prostate cancer, hepatocarcinoma, epithelial carcinoma, non small cell lung cancer or mesothelioma.
